(19) United States
(12) Patent Application Publication
(10) Pub. No.: US 2015/0081226 A1
(43) Pub. Date: Mar. 19, 2015

(54) MANAGING ELECTROENCEPHALOGRAM ANALYSIS

(71) Applicant: Samah G. Abdel Baki, Brooklyn, NY (US)

(72) Inventor: Samah G. Abdel Baki, Brooklyn, NY (US)

(21) Appl. No.: 14/468,528

(22) Filed: Aug. 26, 2014

Related U.S. Application Data
(60) Provisional application No. 61/870,136, filed on Aug. 26, 2013.

(51) Int. Cl. G06F 19/00; A61B 5/0476

(52) U.S. Cl. CPC G06F 19/3418; A61B 5/0476; G06F 19/3431
USPC 702/19

(57) ABSTRACT

Systems and techniques for managing EEG analysis are described herein. EEG data, including an EEG scan of a patient, can be received from a site. The EEG data can be provided to an EEG interpreter. A normalized EEG interpretation for the EEG data can be received from an EEG interpreter. The normalized EEG interpretation can be delivered to the site.

This application claims the benefit of U.S. Provisional Application No. 61/870,136, titled “MANAGING ELECTROENCELPHALOGRAM ANALYSIS,” by Samah G. Abdel Baki, and filed on Aug. 26, 2013, the entire contents of which being incorporated herein by reference.

### TECHNICAL FIELD

Embodiments described herein generally relate to medical devices and more specifically to managing electroencephalogram (EEG) analysis.

### BACKGROUND

EEGs can be a useful tool for diagnosing a variety of cerebral maladies. EEG scans can be complex and difficult to read without proper training Thus, a trained EEG interpreter can be employed to accurately read on EEG scan. When and EEG interpreter is employed, the EEG interpreter can be expected to provide a formal written report of any findings from reading the EEG scan. In an emergency situation, an informal diagnosis can be verbally provided by the EEG interpreter to a medical provider (e.g., attending physician, nurse, etc.) with the formal report to follow.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a system for managing EEG analysis, according to an embodiment.

FIG. 2 is a block diagram of components of an example of a system for EEG analysis, according to an embodiment.

FIGS. 3-6 illustrate example user interfaces for managing EEG analysis, according to an embodiment.

FIG. 7 illustrates an example of a method for managing EEG analysis, according to an embodiment.

FIG. 8 is a block diagram illustrating an example of a machine upon which one or more embodiments may be implemented.

### DETAILED DESCRIPTION

Modern health care demands can strain traditional interactions between medical providers and EEG interpreters. For example, modern regulatory requirements can require a formal report. The time spent creating the informal verbal report and formal written report can represent an inefficiency of time and thus money in the system. Also, for example, an informal report can be of lower quality or less useful to medical providers. This problem can be exacerbated when the EEG interpreter is remote from the patient.

These problems with using EEGs in modern medicine can be ameliorated via management of the EEG analysis environment. For example, structuring the EEG interpretation, for example, via templates (e.g., diagnostic template, form, etc.) to produce a normalized EEG interpretation can save time for the EEG interpreter when crafting the report and can also save time by being be used as the formal report. Further, the normalized EEG interpretation can be used to provide concise information upon which the medical provider may act. Additionally, structuring the raw data (EEG scans, patient data, etc.) from the site can create further efficiencies by allowing EEG interpreters to quickly identify EEG interpretation tasks, provide reports customized to the patient, etc. Additional details of example embodiments are described below.

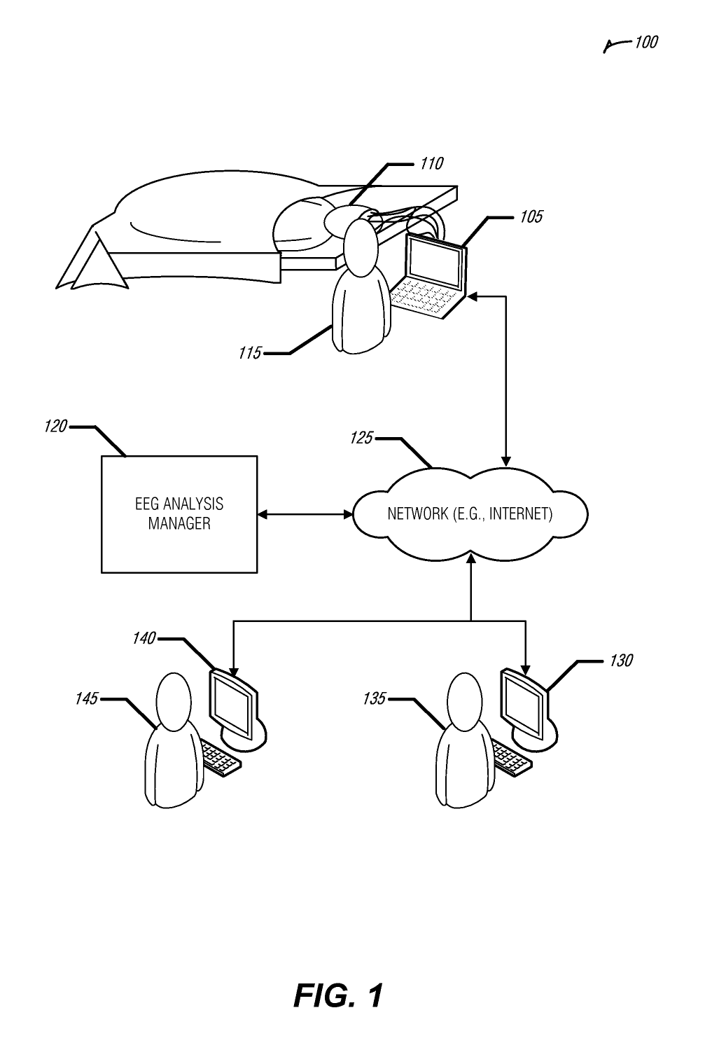

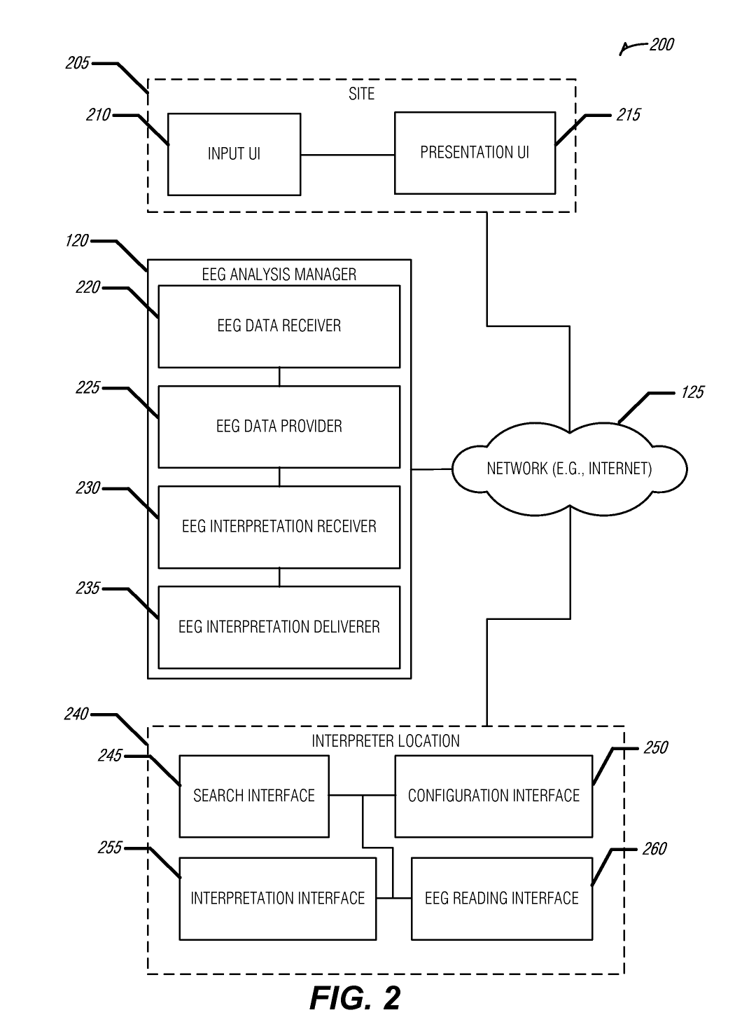

FIG. 1 illustrates an example of an environment 100 for managing EEG analysis. The environment 100 can include an EEG analysis manager 120, described below with respect to FIG. 2. The EEG analysis manager 120 can be communicatively coupled to a site system 105, an EEG interpreter system 130 (e.g., for use by an EEG interpreter 135), or an EEG interpreter reviewer system 140 (e.g., for use by an EEG interpreter reviewer 145) via a network 125.

The site system 105 can include or be connected to apparatus to perform an EEG on a patient 110 and operated by a medical provider 115 (e.g., physician, nurse, etc.) In an example, the site system 105 can be arranged (e.g., configured, structured, manufactured, etc.) to accept data from the medical provider 115. In an example, the site system 105 can be arranged to present data to the medical provider 115. In an example, the site system 105 can be configured to collect or receive situational data. In an example, situational data can include patient data, site data, medical provider data, etc. In an example, patient data can include medical history data, demographic data, diagnostic information for a current patient event (e.g., such as the reason the patient 110 is seeking help from the medical provider 115).

In an example, any or all of the site system 105, EEG interpreter system 130, and the EEG interpreter reviewer system 140 can include software or hardware to present user interfaces to the respective medical provider 115, EEG interpreter 135, and EEG interpreter reviewer 145, that are created or directed from the EEG analysis manager 120. For example, the EEG analysis manager 120 can use a webpage based interface in which the various systems 105, 130, and 140 include a web browser and other attendant hardware and software to present the user interfaces and accept input from the users 115, 135, and 145. If the interfaces are so-called “fat clients,” then the EEG analysis manager 120 can direct these fat clients, residing on the various systems, to present the respective interfaces. In an example, components of the EEG analysis manager 120 can reside on the various systems 105, 130, and 140 in order to provide user interfaces and facilitate data collection.

As here illustrated, an example of managing the EEG analysis can include the site system 105 to perform, or accept, on EEG scan of the patient 110. The site system 105 can present an input interface to the medical provider 115 to add additional information, request a review of the EEG scan, etc. The input interface can originate, or be directed to appear, by the EEG analysis manager 120. The EEG data, including the EEG scan, can be sent via the network 125 to the EEG analysis manager 120. The EEG interpreter 135 can be presented with a search interface to find the EEG data. A normalized (e.g., structured) representation of the EEG data can be presented to the EEG interpreter 135 via an interpretation interface on the EEG interpreter system 130. The interpretation interface can include features to conform acceptable input to a normalized report on the EEG data. In an example, a limited number of diagnostic options can be offered via the interpretation interface to save time and prevent irrelevant data entry. The completed normalized EEG interpretation can be sent back to the EEG analysis manager 120 that can then deliver the normalized EEG interpretation to the site system 105. In an example, the delivered normalized EEG interpretation can be tailored to the medical provider 115, for example, accounting for experience, profession, etc. In an example, the medical provider 115 can use the site system 105 to request a review of the normalized EEG interpretation by the EEG interpretation reviewer 145 via the EEG analysis manager 120 and the EEG interpreter reviewer system 140.

Appendix A includes an example embodiment of the EEG analysis manager 120, called Case Manager.

FIG. 2 is a block diagram of components of an example of a system 200 for EEG analysis. The system 200 can include the EEG analysis manager 120 arranged to be communicatively coupled to a site 205 and interpreter location 240 via the network 125. The EEG analysis manager 120 can include an EEG data receiver 220, an EEG data provider 225, and EEG interpretation receiver 230, and an EEG interpretation deliverer 235. Each of these elements of the EEG analysis manager 120 can be arranged to be communicatively coupled to any one of each other or to the site 205 or interpreter location 240.

The EEG data receiver 220 can be arranged to receive EEG data from the site 205. The EEG data can include an EEG scan of the patient 110. In an example, the EEG data receiver 220 can be arranged to present an input user interface 210 at the site 205. The input user interface 210 can be arranged to accept an EEG context from a user (e.g., medical provider 115) at the site 205. In an example, EEG context can include any information that the user wishes to add to the EEG scan, such as patient name, patient age, circumstances surrounding the EEG scan, etc.

In an example, the EEG data receiver 220 can be arranged to normalize the EEG data. As used throughout this document “normalize(d),” with respect to “normalized EEG data” and “normalized EEG interpretation,” is defined as the structuring of such data in a reduced standard form. For example, if five different EEG interpreters express the equivalent diagnosis in five different ways, the normalized interpretation data will include the single standard way to represent the diagnosis. Normalization can also include directives to the order or placement of data in addition to the acceptable forms and content of the data. In an example, the EEG data receiver can be arranged to normalize the EEG data by combining the EEG context with the EEG scan as the EEG data.

In an example, the EEG data receiver 220 can be arranged to use an input template to normalize the EEG data. In an example, the input template can define a set of data fields and a set of rules corresponding to the set of data fields. Example rules can include specifying a set of data for the data field (e.g., an enum), boundary constraints, other data constraints (e.g., field must be completed, etc.) data mapping (e.g., converting a date written as Aug. 8, 2013 to 20130808), etc. In an example, the set of data fields can include an age of the patient 110. In an example, the data fields can include a diagnosis of the patient. In an example, the diagnosis refers to patient attributes that can be relevant to interpreting the EEG scan. Example attributes can include mental illness, medication, and cerebral injury, among others.

In an example, the input template can be used for the input user interface 210. That is, the input user interface 210 can be arranged by the input template in order to normalize the EEG data. In an example, the input user interface 210 prevents the entry of non-normalized EEG data. This may be accomplished via the input template. In an example, the EEG data receiver 220 can be arranged to select the input template by receiving situational data and selecting the input template (e.g., from a plurality of input templates) based on the situational data. Situational data is similar to EEG context data except that it is gathered from sources other than the input user interface 210. These sources can include admittance system information, patient medical records, medical provider employee system information, medical devices, site sensors, etc. In an example, the situational information can include patient data (e.g., from a medical record system). The input template can be selected based on this patient data. For example, if the patient is a child, the input template can be specific to children. Other situational data that can be relevant includes the state of the patient (e.g., emergency, urgent care, scheduled visit, etc.), state of the facility (e.g., being evacuated, closing, etc.), among others.

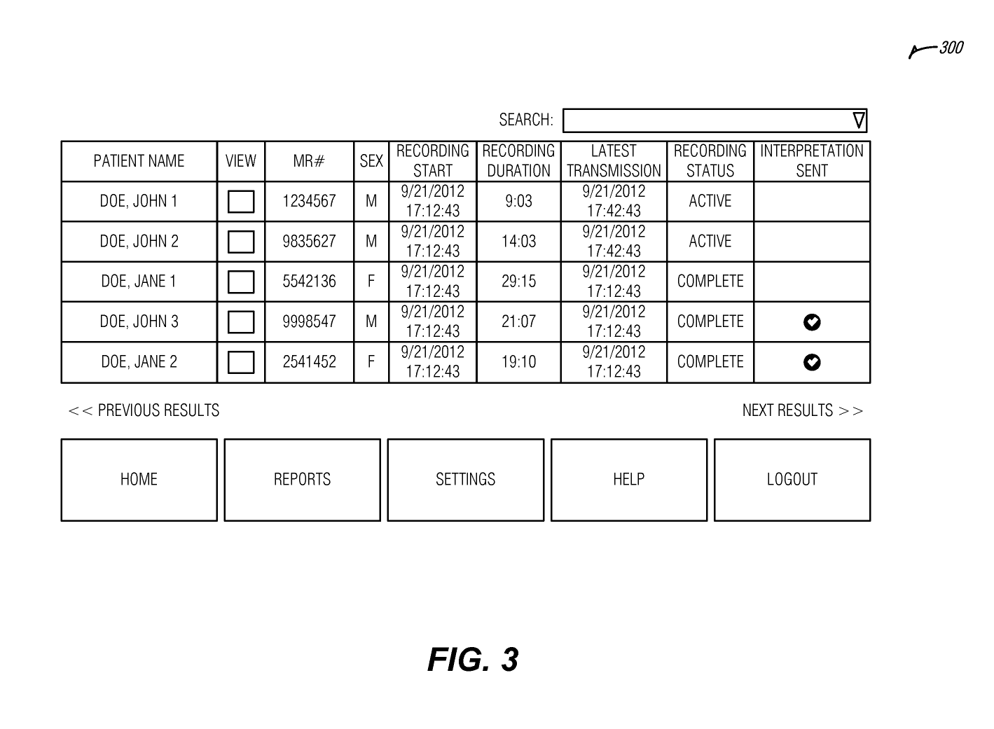

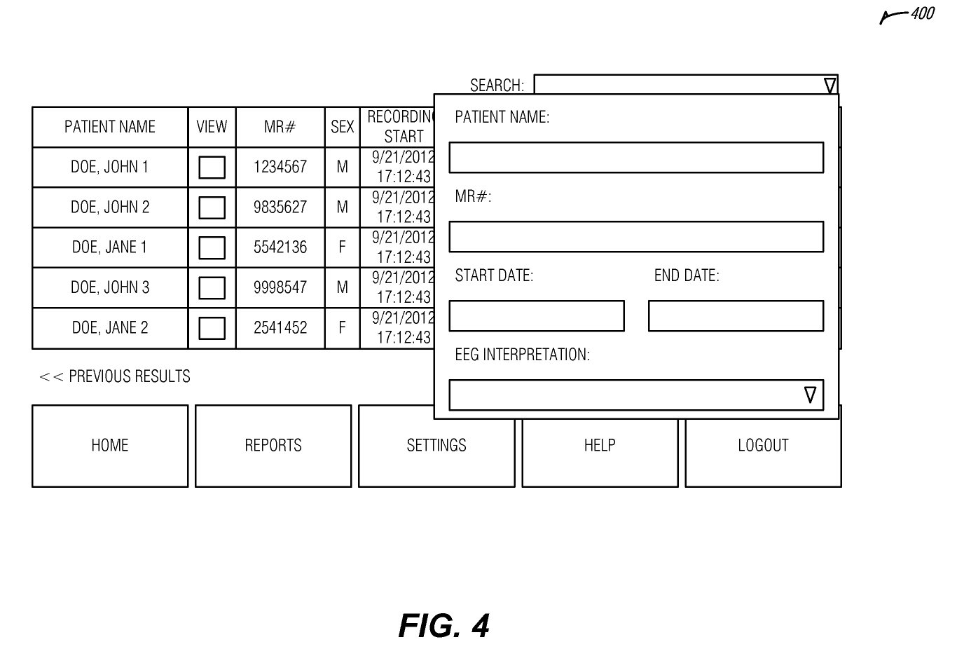

The EEG analysis manager 120 can include an EEG data provider 225 to provide the EEG data to the EEG interpreter 135. In an example, the EEG data provider 225 can be arranged to identify an EEG interpreter 135. In an example, the EEG data provider 225 can be arranged to send a notification, or the EEG data, to the EEG interpreter 135 in response to the identification. In an example, the EEG data provider 225 can be arranged to make the EEG data available to the identified EEG interpreter 135, or to any qualified EEG interpreter. In an example, the EEH data provider 225 can be arranged to present a search interface 245, for example, to the EEG interpreter 135. The EEG data provider 225 can be arranged to accept a search term corresponding to the EEG data from the EEG interpreter 135 via the search interface 245. The EEG data provider 225 can be arranged to present a search result interface in response to receiving the search term. In an example, the search result interface can include a representation of the EEG data. FIGS. 3 and 4 below include examples of user interfaces for the search interface 245 and search result interface. In an example, the representation of the EEG data can include information from the EEG data that uniquely identifies the EEG scan. Such data can include a patient name or number, a medical record number, time, date, medical provider identification, etc. In an example, the searchable terms can be defined by the normalized EEG data.

In an example, the EEG data provider 225 can be arranged to provide an EEG reading interface 260. The EEG reading interface 260 can be arranged to represent the EEG scan to the EEG interpreter 135. Thus, the EEG interpreter does not require specific software or hardware to read the EEG scan at the EEG interpreter system 130. Example implementations of the EEG reading interface 260 can include a conversion of a proprietary EEG scan format to a video, an interactive application, animation, etc. by the EEG data provider 225. The EEG reading interface 260 can be arranged to show, run, etc., the converted EEG scan to the EEG interpreter 135. In an example, the EEG reading interface 260 can be arranged to accept annotations of the EEG scan by the EEG interpreter. Accepting annotations can include a graphical interface by which a portion of the scan can be highlighted, a point in the scan can be clicked, or other features of the EEG scan can be identified by the EEG interpreter 135. Accepting annotations can also include data fields to specify the start and end periods of the scan to which the annotation is attached. Annotations can also include interpretation data. Thus, the EEG interpreter 135 can specify to which portions of the EEG scan the EEG interpretation pertains. In an example, the annotations can be included into the normalized EEG interpretation.

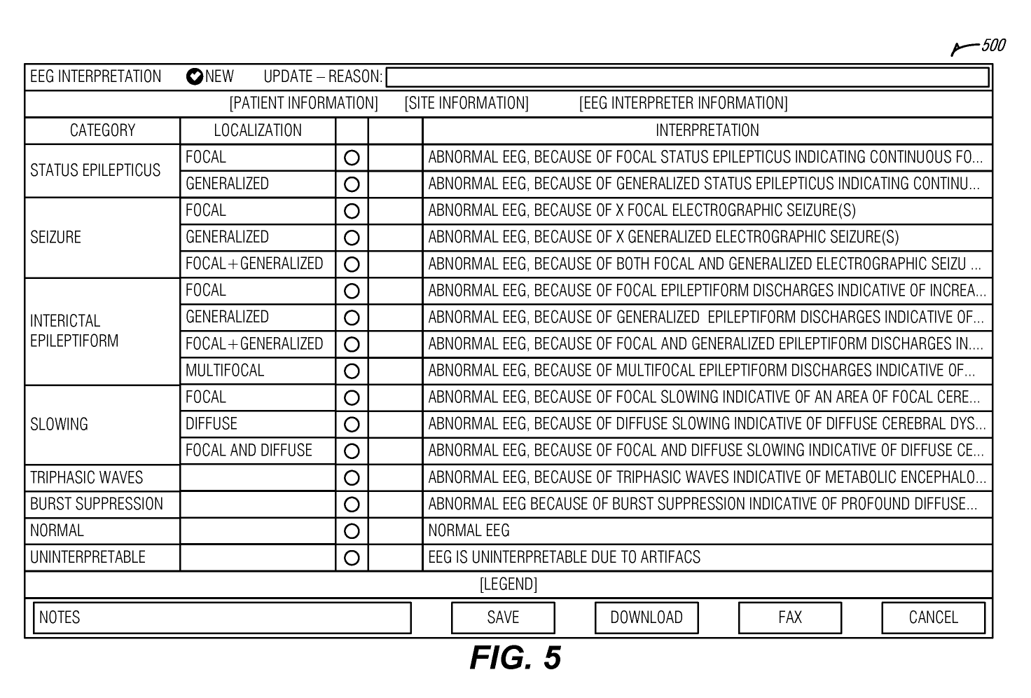

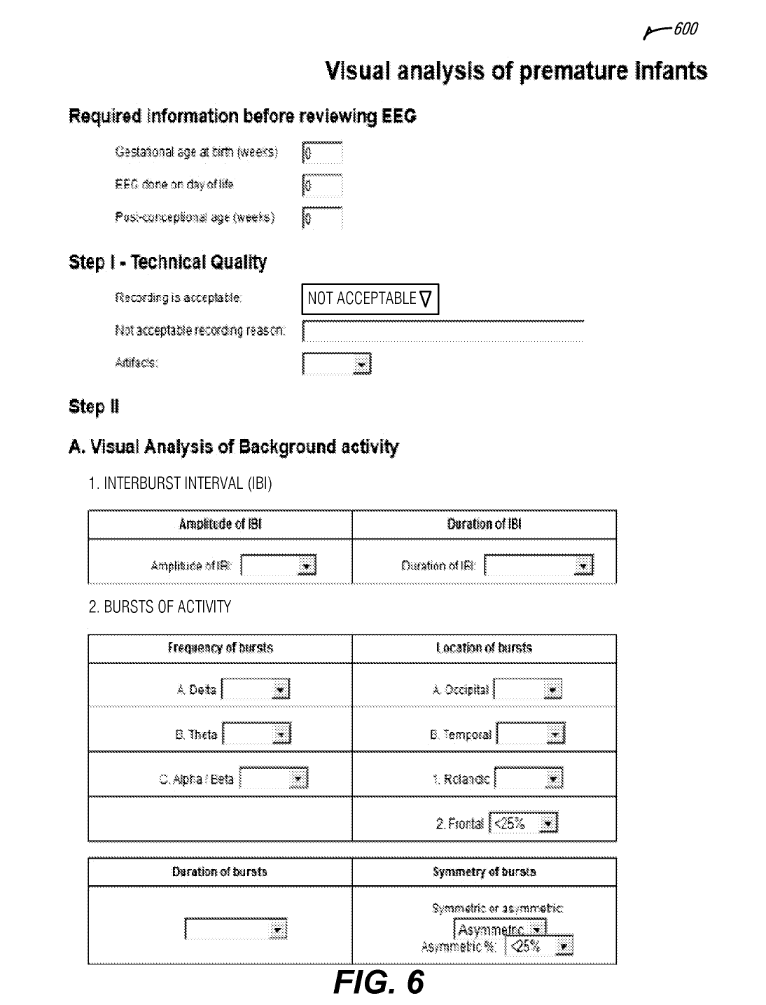

In an example, the EEG data provider can be arranged to present an interpretation interface 225 to the EEG interpreter 135. In an example, the EEG interpretation interface 255 can be combined with the EEG reading interface 260. In an example, the interpretation interface 255 can include a set of pre-defined data entry fields. These fields can ensure that only normalized data is accepted from the EEG interpreter 135. Further, these fields can speed authoring of the EEG interpretation by freeing the EEG data interpreter 135 from entering redundant data, but rather allowing the EEG interpreter 135 to select appropriate answers. FIG. 5 below illustrates one such interpretation interface. In an example, the pre-defined data entry fields can be defined in an interpretation template. In an example, the EEG data provider 225 can be arranged to select the interpretation template from a set of interpretation templates. In an example, this selecting can be based on a patient differentiator in the EEG data. A patient differentiator can be any EEG data that differentiates one patient from another with respect to the EEG interpretation. IN an example, the patient differentiator is an age of the patient 110. FIGS. 5 and 6 illustrate two examples of interpretation interfaces 255 that can be based on templates with a patient differentiator of age. Thus, for an adult patient, the interpretation interface 500 can be presented to the EEG interpreter 135 and for a premature infant (or other pediatric), interpretation interface 600 can be presented. In an example, the selected template can correspond to the EEG interpreter 135. Thus, different EEG interpreters can receive different interpretation templates with all else being equal. This can increase individual EEG interpreter efficiency if, for example, different data entry layouts work better for different EEG interpreters.

In an example, the interpretation template (or other templates) can include layout information for at least one data entry field. The layout information can dictate the positioning of the data entry field in the interpretation interface 255. Such information can allow for the reorganization of data entry for more optimal time savings at a later date. Further, layout information can include “TAB” or other transition information indicating which field a keystroke, for example, will cause the data input focus to shift to. In an example, the EEG analysis manager 120 can include a configuration module arranged to allow a user, such as the EEG interpreter 135, medical provider 115, or system administrator, to edit templates, including the layout information. In an example, the configuration module be arranged to present a configuration interface 250, receive data entry field information or layout information via the configuration interface 250, and update the template with the received information.

The EEG interpretation receiver 230 can be arranged to receive a normalized EEG interpretation for the EEG data from the EEG interpreter 135. In an example, the EEG interpretation receiver 230 can be arranged to affix an electronic signature of the EEG interpreter 135 to the normalized EEG interpretation. In an example, the interpretation interface 255 can be arranged to accept an indication by the EEG interpreter 135 that the normalized EEG interpretation is ready for signing. In an example, the EEG interpretation receiver 230 can be arranged to verify an electronic signature affixed to the normalized EEG interpretation by the interpretation interface 255. In an example, the EEG interpretation receiver 230 can be arranged to verify that the EEG interpretation data is normalized.

In an example, the EEG interpretation receiver 230 can be arranged to deliver the normalized EEG interpretation to a set of recipients. Such a distribution can be used to satisfy reporting or record keeping requirements without additional work by either the medical provider 115 or the EEG interpreter 135.

The EEG interpretation deliverer 235 can be arranged to deliver the normalized EEG interpretation to the site 205. In an example, the EEG interpretation deliverer 235 can be arranged to present a presentation user interface 215 at the site 205 (e.g., via the site system 105). In an example, the presentation user interface 215 can be arranged to present a targeted presentation of the normalized EEG interpretation. In an example, the targeted presentation can be targeted as a particular medical provider 115 or class of medical providers (e.g., physician, nurse, hospital administrator, etc.). In an example, the EEG interpretation deliverer 235 can be arranged to identify the target medical provider and adjust a detail level of the normalized EEG interpretation based on the identified target medical provider. For example, if the target medical provider is a nurse, the detail level of the normalized EEG interpretation can be reduced to indicate an immediate intervention is necessary and that appropriate personal should be notified. If the target medical provider is the attending physician, the detail level can be appropriate for the physician to act on. Thus, the formal details of the EEG interpretation can be maintained while an audience specific form can be used to achieve the greatest effect at the site 205.

In an example, the EEG interpretation deliverer 235 can be arranged to send a report of the normalized EEG interpretation via a communication conduit selected for compliance with a reporting standard. Although the normalized EEG interpretation can be a formal written report (e.g., including those electronically signed by the EEG interpreter 135), in some instances specific delivery mechanisms may also be mandated. Communication conduits can include email, facsimile transmission (e.g., a fax), printed and delivered, etc., as opposed to being delivered, for example, via the presentation user interface 215. In an example, the EEG interpretation deliverer 235 can be arranged to deliver the report to an electronic medical record system of the patient 110. In an example, the EEG interpretation deliverer 235 can be arranged to update the electronic medical record of the patient 110 directly with the report.

In an example, the EEG interpretation deliverer 235 can be arranged to deliver the normalized EEG interpretation, and in some cases the EEG data, to a second EEG interpreter, such as the EEG interpreter reviewer 145. This action may be performed in response to receiving, from the site 205, a review indication corresponding to the normalized EEG interpretation. Thus, the medical provider 115 can ask for a second opinion via the presentation user interface 215. The workflow and options for the EEG interpreter reviewer as managed by the system 200 can include every feature discussed above with respect to the EEG interpreter 135. In an example, additional data can be included to facilitate review, such as identification of the EEG interpreter, user interface elements to permit simple judgment as to correctness of the normalized EEG interpretation, etc.

FIGS. 3-6 illustrate example user interfaces 300-600 for managing EEG analysis.

FIG. 3 illustrates an example of a user interface 300 including a combined search interface and search result interface. In this example, the search interface can be invoked via the labeled textbox or the pull-down arrow in that text box. The search result interface includes a variety of fields, including a view field in which the EEG scan may be viewed. As used herein, “MR#” refers to a “medical record number.”

FIG. 4 illustrates an example of a user interface 400 that is similar to the user interface 300. The user interface 400 additionally includes a more structured search interface invoked, for example, by clicking on the pull-down arrow in the search textbox.

FIG. 5 illustrates an example of an interpretation interface 500. In this example, EEG interpretation normalization is enforced via the input mechanism. For example, the EEG interpretation is a marking of a circle (and in some examples more than one circle) corresponding to a category and localization combination. In an example, the interpretation (represented on the right) is immutable. Thus, the interpretation represents the conical form of the diagnosis. This interpretation interface 500 can be specifically crafted or configurable via templating. In an example, the interpretation interface 500 is for an adult.

FIG. 6 illustrates an example of an interpretation interface 600 for premature infants. Other interpretation interfaces, or those arranged by templates, can be used for specific groups of people, for example, differentiated by age, gender, medical diagnosis (e.g., mental injury, disease, etc.), among others.

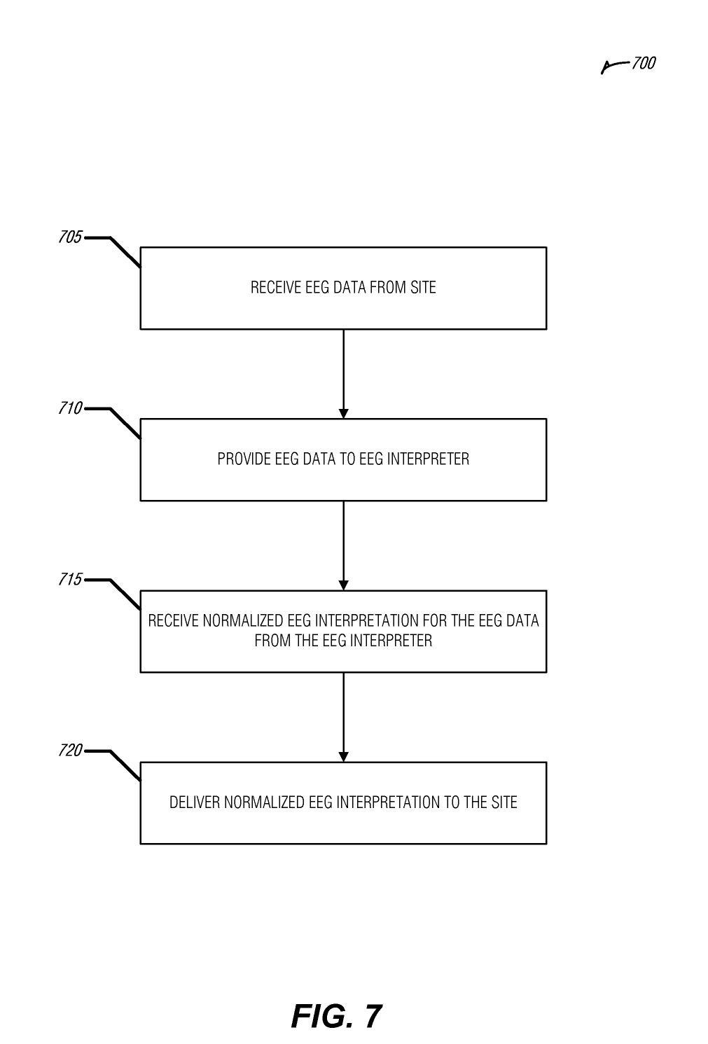

FIG. 7 illustrates an example of a method 700 for managing EEG analysis. Operations of the method 700 can be performed by any computational hardware (e.g., processors, circuits, etc.), such as the various components discussed above with respect to FIGS. 1-6.

At operation 705, EEG data can be received from a site. The EEG data can include an EEG scan of a patient. In an example, the operation 705 can include presenting an input user interface arranged to accept EEG context from a user at the site. Further, the operation 705 can include normalizing the EEG data including combining the EEG scan and the EEG context as the EEG data.

In an example, normalizing the EEG data can include using an input template for the input user interface. The input template can define a set of data fields and a set of rules corresponding to the set of data fields. In an example, the set of data fields can include an age of the patient. In an example, the set of data fields can include a diagnosis of the patient. In an example, the rules can include a set of acceptable values for the respective data fields. In an example, the set of acceptable values can include a set with no empty (e.g., null, blank, etc.) members.

In an example, using the input template can include selecting the input template from a set of input templates. In an example, selecting the input template can include receiving situational data and select the input template based on the situational data. In an example, the situational data can include patient data. In an example, selecting the input template can include using the patient data. Situational data can differ from EEG context data in that the EEG context data is entered by a user and the situational data can be determined from medical records, sensors, or other automatic data mechanisms.

At operation 710, the EEG data can be provided to an EEG interpreter. In an example, providing the EEG data to the EEG interpreter can include presenting a search interface (e.g., to the EEG interpreter), accepting a search term corresponding to the EEG data via the search interface, and presenting a search result interface to the EEG interpreter in response to the search term. The search result interface can include a representation of the EEG data.

In an example, providing the EEG data to the EEG interpreter can include providing an EEG reading interface (e.g., to the EEG interpreter). The EEG reading interface can be arranged to represent the EEG scan to the EEG interpreter. For example, the EEG scan can be converted into a common format video file or other format available on the EEG interpreter system 130. Thus, no special EEG scan reading software is required by the EEG interpreter in order to view the EEG scan. In an example, the EEG reading interface can be arranged to accept annotations of the EEG scan by the EEG interpreter and include the annotations in the normalized EEG interpretation. In this example, annotations can include marking periods of the scan, or features of the scan, and attaching data to the markings. In an example, the normalized EEG interpretation can refer to a set of annotations to which it pertains.

In an example, providing the EEG data to the EEG interpreter can include presenting an interpretation interface with a set of pre-defined data entry fields. In an example, the pre-defined data entry fields can be defined in an interpretation template. In an example, presenting the interpretation interface can include selecting the interpretation template from a set of interpretation templates based on a patient differentiator in the EEG data. In an example, the patient differentiator can be an age of the patient. In an example, the age can be represented as a category, such as adult, child, neonatal, etc.

In an example, the interpretation template can include layout information for a data entry field. The interpretation interface can also include positioning the data entry field in accordance with the layout information. In an example, the interpretation template can correspond to the EEG interpreter and the interpretation interface can include selecting the interpretation template from the set of interpretation templates based on the EEG interpreter. In an example, the EEG interpreter can configure the layout of an interpretation template (e.g., a personalized template). To facilitate this, a configuration interface can be presented. Layout information for the data entry field can be received via the configuration interface. The template can be updated with the received layout information. In this manner, the EEG interpreter can rearrange the data entry fields, if desired, into a more comfortable or efficient layout for data entry.

At operation 715, a normalized EEG interpretation for the EEG data can be received from the EEG interpreter. In an example, receiving the normalized EEG interpretation can include affixing an electronic signature of the EEG interpreter to the normalized EEG interpretation. In an example, receiving the normalized EEG interpretation can include delivering the normalized EEG interpretation to a set of recipients. The set of recipients can include a member that is not the site. Thus, for example, reporting partners can be included in the EEG analysis without imposing additional burdens on either the EEG interpreter or the medical provider.

At operation 720, the normalized EEG interpretation can be delivered to the site. In an example, delivering the normalized EEG interpretation to the site can include presenting a targeted presentation of the normalized EEG interpretation. In an example, presenting the targeted presentation can include identifying a target medical provider and adjusting a detail level of the normalized EEG interpretation based on the target medical provider.

In an example, delivering the normalized EEG interpretation to the site can include sending a report of the normalized EEG interpretation via a communication conduit selected for compliance with a reporting standard. In an example, delivering the normalized EEG interpretation can include sending the report to an electronic medical record system of the patient.

In an example, delivering the normalized EEG interpretation to the site can include providing the EEG data and the normalized EEG interpretation to a second EEG interpreter in response to receiving, from the site, a review indication corresponding to the normalized EEG interpretation. Thus, a user can request a second opinion on an EEG interpretation.

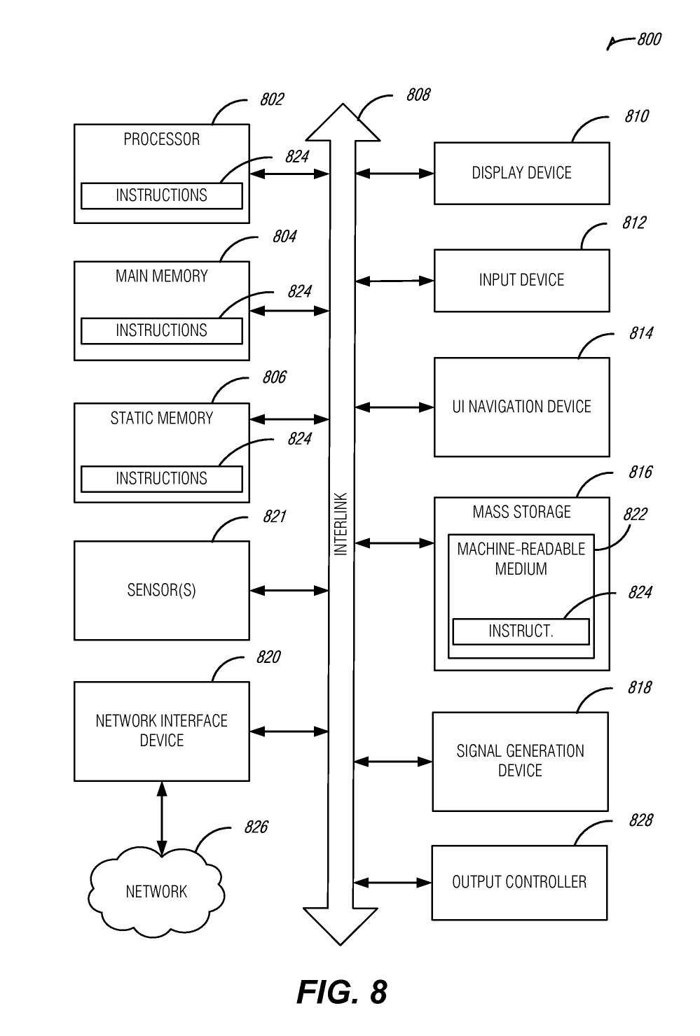

FIG. 8 illustrates a block diagram of an example machine 800 upon which any one or more of the techniques (e.g., methodologies) discussed herein may perform. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, device partitions, or mechanisms. Modules are tangible entities (e.g., hardware) capable of performing specified operations and may be arranged or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be arranged by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically arranged (e.g., hardwired), or temporarily (e.g., transitorily) arranged (e.g., programmed) to operate in a specified manner or to perform part or all of any operation described herein. Considering examples in which modules are temporarily arranged, each of the modules need not be instantiated at any one moment in time. For example, where the modules comprise a general-purpose hardware processor arranged using software, the general-purpose hardware processor may be arranged as respective different modules at different times. Software may accordingly configure a hardware processor, for example, to constitute a particular module at one instance of time and to constitute a different module at a different instance of time.

A device partition is a collection of hardware elements or portions of elements that are structured to perform a function. For example, a set of analog signal processing elements for radio communications can be a RF communications partition for modulating or demodulating RF communications. Further, a set of digital elements arranged to be communicatively coupled (e.g., when the device is powered on) to a machine readable medium with instructions to configure the digital elements to modulate or demodulate radio communications can also be a RF communications partition. Thus, a device partition always includes device hardware.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine readable media.

While the machine readable medium 822 is illustrated as a single medium, the term “machine readable medium” may include a single medium or multiple media (e.g., a centralized or distributed database, and/or associated caches and servers) arranged to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, and optical and magnetic media. In an example, a machine readable medium is not a transitory propagating signal. In an example, a massed machine readable medium comprises a machine readable medium with a plurality of particles having invariant (e.g., resting) mass. Thus, a massed machine readable medium is not a transitory propagating signal. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

### Additional Notes & Examples

Although expressed in singly dependent form, the claims represent embodiments that can be combined in any way. Thus, for example, claim 10 may dependent from any one or more of claims 1-9.

The above detailed description includes references to the accompanying drawings, which form a part of the detailed description. The drawings show, by way of illustration, specific embodiments that may be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A machine-readable media that are not a transitory propagating signal, the machine-readable media including instructions that, when executed by a machine system, cause the machine system to perform operations comprising:
receiving EEG data from a site, the EEG data including an EEG scan of a patient;
providing the EEG data to an EEG interpreter;
receiving a normalized EEG interpretation for the EEG data from the EEG interpreter; and
delivering the normalized EEG interpretation to the site.

2. The machine-readable media of claim 1, wherein receiving the EEG data from the site includes:
presenting an input user interface arranged to accept an EEG context from a user at the site; and
normalizing the EEG data including combining the EEG scan and the EEG context as the EEG data.

3. The machine-readable media of claim 2, wherein normalizing the EEG data includes using an input template for the input user interface, the input template defining a set of data fields and a set of rules corresponding to the set of data fields.

4. The machine-readable media of claim 3, wherein the set of data fields includes an age of the patient.

5. The machine-readable media of claim 3, wherein the set of data fields includes a diagnosis of the patient.

6. The machine-readable media of claim 3, wherein using the input template includes selecting the input template from a set of input templates.

7. The machine-readable media of claim 6, wherein selecting the input template includes:
receiving situational data; and
selecting the input template based on the situational data.

8. The machine-readable media of claim 7, wherein the situational data includes patient data, and wherein selecting the input template based on the situational data includes using the patient data.

9. The machine-readable media of claim 1, wherein delivering the normalized EEG interpretation to the site includes presenting a targeted presentation of the normalized EEG interpretation.

10. The machine-readable media of claim 9, wherein presenting the targeted presentation includes:
identifying a target medical provider; and
adjusting a detail level of the normalized EEG interpretation based on the target medical provider.

11. The machine-readable media of claim 1, wherein delivering the normalized EEG interpretation to the site includes sending a report of the normalized EEG interpretation via a communication conduit selected for compliance with a reporting standard.

12. The machine-readable media of claim 11, wherein delivering the normalized EEG interpretation includes sending the report to an electronic medical record system of the patient.

13. The machine-readable media of claim 1, wherein delivering the normalized EEG interpretation to the site includes providing the EEG data and the normalized EEG interpretation to a second EEG interpreter in response to receiving, from the site, a review indication corresponding to the normalized EEG interpretation.

14. The machine-readable media of claim 1, wherein providing the EEG data to the EEG interpreter includes:
presenting a search interface;
accepting a search term corresponding to the EEG data from the EEG interpreter via the search interface;
presenting a search result interface to the EEG interpreter including a representation of the EEG data.

15. The machine-readable media of claim 1, wherein providing the EEG data to the EEG interpreter includes providing an EEG reading interface, the EEG reading interface arranged to represent the EEG scan to the EEG interpreter.

16. The machine-readable media of claim 15, wherein the EEG reading interface is arranged to accept annotations of the EEG scan by the EEG interpreter and include the annotations in the normalized EEG interpretation.

17. The machine-readable media of claim 1, wherein providing the EEG data to the EEG interpreter includes presenting an interpretation interface with a set of pre-defined data entry fields.

18. The machine-readable media of claim 17, wherein the pre-defined data entry fields are defined in an interpretation template.

19. The machine-readable media of claim 18, wherein presenting the interpretation interface includes selecting the interpretation template from a set of interpretation templates based on a patient differentiator in the EEG data.

20. The machine-readable media of claim 19, wherein the patient differentiator is an age of the patient.

21. The machine-readable media of claim 18, wherein the template includes layout information for a data entry field, and wherein presenting the interpretation interface includes positioning the data entry field in accordance with the layout information.

22. The machine-readable media of claim 21 including instructions comprising:
presenting a configuration interface;
receiving the layout information for the data entry field via the configuration interface; and
updating the template with the layout information.

23. The machine-readable media of claim 21, wherein the template corresponds to the EEG interpreter, and wherein presenting the interpretation interface includes selecting the interpretation template from a set of interpretation templates based on the EEG interpreter.

24. The machine-readable media of claim 1, wherein receiving the normalized EEG interpretation includes affixing an electronic signature of the EEG interpreter to the normalized EEG interpretation.

25. The machine-readable media of claim 1, wherein receiving the normalized EEG interpretation includes delivering the normalized EEG interpretation to a set of recipients, the set of recipients including a member that is not the site.

26. A system for managing electroencephalogram (EEG) analysis, the system comprising:
an EEG data receiver to receive EEG data from a site, the EEG data including an EEG scan of a patient;
an EEG data provider to provide the EEG data to an EEG interpreter;
an EEG interpretation receiver to receive a normalized EEG interpretation for the EEG data from the EEG interpreter; and
an EEG interpretation deliverer to deliver the normalized EEG interpretation to the site.

27. The system of claim 26, wherein to receive the EEG data from the site includes the EEG data receiver to:
present an input user interface arranged to accept an EEG context from a user at the site; and
normalize the EEG data including combining the EEG scan and the EEG context as the EEG data.

28. The system of claim 27, wherein to normalize the EEG data includes the EEG data receiver to use an input template for the input user interface, the input template defining a set of data fields and a set of rules corresponding to the set of data fields.

29. The system of claim 28, wherein the set of data fields includes an age of the patient.

30. The system of claim 28, wherein the set of data fields includes a diagnosis of the patient.

31. The system of claim 28, wherein to use the input template includes the EEG data receiver to select the input template from a set of input templates.

32. The system of claim 31, wherein to select the input template includes the EEG data receiver to:
receive situational data; and
select the input template based on the situational data.

33. The system of claim 32, wherein the situational data includes patient data, and wherein to select the input template based on the situational data includes the EEG data receiver to use the patient data.

34. The system of claim 26, wherein to deliver the normalized EEG interpretation to the site includes the EEG interpretation deliverer to present a targeted presentation of the normalized EEG interpretation.

35. The system of claim 34, wherein to present the targeted presentation includes the EEG interpretation deliverer to:
identify a target medical provider; and
adjust a detail level of the normalized EEG interpretation based on the target medical provider.

36. The system of claim 26, wherein to deliver the normalized EEG interpretation to the site includes the EEG interpretation deliverer to send a report of the normalized EEG interpretation via a communication conduit selected for compliance with a reporting standard.

37. The system of claim 36, wherein to deliver the normalized EEG interpretation includes the EEG interpretation deliverer to send the report to an electronic medical record system of the patient.

38. The system of claim 26, wherein to deliver the normalized EEG interpretation to the site includes the EEG interpretation deliverer to provide the EEG data and the normalized EEG interpretation to a second EEG interpreter in response to receiving, from the site, a review indication corresponding to the normalized EEG interpretation.

39. The system of claim 26, wherein to provide the EEG data to the EEG interpreter includes the EEG data provider to:
present a search interface;
accept a search term corresponding to the EEG data from the EEG interpreter via the search interface;
present a search result interface to the EEG interpreter including a representation of the EEG data.

40. The system of claim 26, wherein to provide the EEG data to the EEG interpreter includes the EEG data provider to provide an EEG reading interface, the EEG reading interface arranged to represent the EEG scan to the EEG interpreter.

41. The system of claim 40, wherein the EEG reading interface is arranged to accept annotations of the EEG scan by the EEG interpreter and include the annotations in the normalized EEG interpretation.

42. The system of claim 26, wherein to provide the EEG data to the EEG interpreter includes the EEG data provider to present an interpretation interface with a set of pre-defined data entry fields.

43. The system of claim 42, wherein the pre-defined data entry fields are defined in an interpretation template.

44. The system of claim 43, wherein to present the interpretation interface includes the EEG data provider to select the interpretation template from a set of interpretation templates based on a patient differentiator in the EEG data.

45. The system of claim 44, wherein the patient differentiator is an age of the patient.

46. The system of claim 43, wherein the template includes layout information for a data entry field, and wherein to present the interpretation interface includes the EEG data provider to position the data entry field in accordance with the layout information.

47. The system of claim 46 comprising a configuration module to:
present a configuration interface;
receive the layout information for the data entry field via the configuration interface; and
update the template with the layout information.

48. The system of claim 46, wherein the template corresponds to the EEG interpreter, and wherein to present the interpretation interface includes the EEG data provider to select the interpretation template from a set of interpretation templates based on the EEG interpreter.

49. The system of claim 26, wherein to receive the normalized EEG interpretation includes the EEG interpretation receiver to affix an electronic signature of the EEG interpreter to the normalized EEG interpretation.

50. The system of claim 26, wherein to receive the normalized EEG interpretation includes the EEG interpretation receiver to deliver the normalized EEG interpretation to a set of recipients, the set of recipients including a member that is not the site.

51. A method for managing electroencephalogram (EEG) analysis, the method comprising:
receiving EEG data from a site, the EEG data including an EEG scan of a patient;
providing the EEG data to an EEG interpreter;
receiving a normalized EEG interpretation for the EEG data from the EEG interpreter; and
delivering the normalized EEG interpretation to the site.

52. The method of claim 51, wherein receiving the EEG data from the site includes:
presenting an input user interface arranged to accept an EEG context from a user at the site; and
normalizing the EEG data including combining the EEG scan and the EEG context as the EEG data.

53. The method of claim 52, wherein normalizing the EEG data includes using an input template for the input user interface, the input template defining a set of data fields and a set of rules corresponding to the set of data fields.

54. The method of claim 53, wherein the set of data fields includes an age of the patient.

55. The method of claim 53, wherein the set of data fields includes a diagnosis of the patient.

56. The method of claim 53, wherein using the input template includes selecting the input template from a set of input templates.

57. The method of claim 56, wherein selecting the input template includes:
receiving situational data; and
selecting the input template based on the situational data.

58. The method of claim 57, wherein the situational data includes patient data, and wherein selecting the input template based on the situational data includes using the patient data.

59. The method of claim 51, wherein delivering the normalized EEG interpretation to the site includes presenting a targeted presentation of the normalized EEG interpretation.

60. The method of claim 59, wherein presenting the targeted presentation includes:
identifying a target medical provider; and
adjusting a detail level of the normalized EEG interpretation based on the target medical provider.

61. The method of claim 51, wherein delivering the normalized EEG interpretation to the site includes sending a report of the normalized EEG interpretation via a communication conduit selected for compliance with a reporting standard.

62. The method of claim 61, wherein delivering the normalized EEG interpretation includes sending the report to an electronic medical record system of the patient.

63. The method of claim 51, wherein delivering the normalized EEG interpretation to the site includes providing the EEG data and the normalized EEG interpretation to a second EEG interpreter in response to receiving, from the site, a review indication corresponding to the normalized EEG interpretation.

64. The method of claim 51, wherein providing the EEG data to the EEG interpreter includes:
presenting a search interface;
accepting a search term corresponding to the EEG data from the EEG interpreter via the search interface;
presenting a search result interface to the EEG interpreter including a representation of the EEG data.

65. The method of claim 51, wherein providing the EEG data to the EEG interpreter includes providing an EEG reading interface, the EEG reading interface arranged to represent the EEG scan to the EEG interpreter.

66. The method of claim 65, wherein the EEG reading interface is arranged to accept annotations of the EEG scan by the EEG interpreter and include the annotations in the normalized EEG interpretation.

67. The method of claim 51, wherein providing the EEG data to the EEG interpreter includes presenting an interpretation interface with a set of pre-defined data entry fields.

68. The method of claim 67, wherein the pre-defined data entry fields are defined in an interpretation template.

69. The method of claim 68, wherein presenting the interpretation interface includes selecting the interpretation template from a set of interpretation templates based on a patient differentiator in the EEG data.

70. The method of claim 69, wherein the patient differentiator is an age of the patient.

71. The method of claim 68, wherein the template includes layout information for a data entry field, and wherein presenting the interpretation interface includes positioning the data entry field in accordance with the layout information.

72. The method of claim 71 comprising:
presenting a configuration interface;
receiving the layout information for the data entry field via the configuration interface; and
updating the template with the layout information.

73. The method of claim 71, wherein the template corresponds to the EEG interpreter, and wherein presenting the interpretation interface includes selecting the interpretation template from a set of interpretation templates based on the EEG interpreter.

74. The method of claim 51, wherein receiving the normalized EEG interpretation includes affixing an electronic signature of the EEG interpreter to the normalized EEG interpretation.

75. The method of claim 51, wherein receiving the normalized EEG interpretation includes delivering the normalized EEG interpretation to a set of recipients, the set of recipients including a member that is not the site.
